(19) United States
(12) Patent Application Publication
(10) Pub. No.: US 2003/0013189 A1
(43) Pub. Date: Jan. 16, 2003

(54) Compositions and methods useful for non-invasive delivery of therapeutic molecules to the bloodstream

(76) Inventors: James M. Wilson, Gladwyne, PA (US); Alberto Auricchio, Philadelphia, PA (US); Markus Hildinger, Evanston, IL (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 09/955,444

(22) Filed: Sep. 17, 2001

Related U.S. Application Data
(63) Continuation-in-part of application No. PCT/US01/13000, filed on Apr. 23, 2001.
(60) Provisional application No. 60/200,409, filed on Apr. 28, 2000.

(51) Int. Cl. A61K 39/12; A61K 39/23; A61K 39/235; C12N 7/00; C12N 7/01; A61K 45/00; A61K 47/00; C12N 15/00; C12N 15/09; C12N 15/63; C12N 15/70; C12N 15/74

(52) U.S. Cl. 435/320.1; 435/235.1; 424/199.1; 424/233.1; 424/278.1

(57) ABSTRACT

A non-invasive method for obtaining therapeutic levels of protein in the bloodstream by administering rAAV containing a transgene encoding the secretable or extracellular membrane-bound protein via inhalation, is provided. Suitably, the transgene product is under the control of a lung-specific promoter. Also provided are pharmaceutical kits containing rAAV encoding the secretable protein in a suspension suitable for delivery via intranasal or oral inhalation.

### CROSS-REFERENCE TO RELATED APPLICATIONS

This is a continuation-in-part of International Patent Application No. PCT/US01/13000, filed Apr. 23, 2001, which claims the benefit of U.S. patent application Ser. No. 60/200,409, filed Apr. 28, 2000, which are incorporated by reference herein.

This work was funded, in part, by a grant from the National Institutes of Health (NIH) P30 DK47757-07 and P01 HL59407-02. The U.S. government has certain rights in this invention.

### BACKGROUND OF THE INVENTION

The invention relates generally to the field of viral vectors useful in gene delivery.

There are a variety of therapeutic and immunogenic molecules for which delivery to the blood is desirable. Such molecules include those useful for treatment of blood disorders, e.g., hemophilia, and molecules useful for cancer therapies, conferring passive immunity, and a variety of other purposes. However, current methods for delivery of such molecules to the blood via viral vectors involve injection, or other highly invasive methods, which require delivery by health care professionals.

What is needed in the art are novel methods for delivering therapeutic and immunogenic molecules to the blood.

### SUMMARY OF THE INVENTION

In one aspect, the invention provides a non-invasive method for obtaining therapeutic levels of protein in the bloodstream. The method involves administering to a subject, by inhalation, a recombinant adeno-associated virus (rAAV) containing a transgene encoding a secreted or extracellular membrane-bound protein.

In another aspect, the invention provides a pharmaceutical kit for delivery of a product. The kit may contain a container for administration of a predetermined dose by inhalation. The kit further contains a suspension containing the recombinant AAV for aerosol or spray delivery of a predetermined dose by inhalation, said suspension comprising a rAAV comprising a transgene encoding a secreted or membrane-bound product and a physiologically compatible carrier.

Other aspects and advantages of the invention will be readily apparent to one of skill in the art from the detailed description of the invention.

### BRIEF DESCRIPTION OF THE DRAWINGS

FIG. 1 illustrates serum erythropoietin levels (mU/mL) at various time points following intranasal delivery of rAAV2/5 vectors carrying either lacZ or recombinant human erythropoietin (rhEpo). See, Example 3.

### DETAILED DESCRIPTION OF THE INVENTION

The present invention provides a non-invasive route of administration for delivering heterologous molecules to the bloodstream at therapeutic levels via recombinant AAV vectors. This non-invasive route of administration is advantageous because it allows for management of therapeutic regimens at home, better patient compliance, and therefore, a higher success rate of therapy.

I. Non-invasive Methods of Delivering Heterologous Molecules to the Bloodstream

In one desirable embodiment, the invention provides a method for AAV-mediated delivery of a heterologous molecule to a host by inhalation.

As used herein, a heterologous molecule may be any substance which is desired to be delivered to a cell, including, without limitation, a polypeptide, protein, enzyme, carbohydrate, chemical moiety, or nucleic acid sequences which may include oligonucleotides, RNA, and/or DNA. The heterologous molecule carried by the rAAV for delivery to the bloodstream are such molecules as defined herein which are secreted by the cell to which they are delivered, or are expressed on the outside of the cell membrane, and are passed into the bloodstream.

As used herein, a transgene is a nucleic acid sequence which encodes a polypeptide, protein, enzyme or other product of interest operatively linked to regulatory components in a manner which permits transcription, translation and/or ultimately directs expression of a product encoded by the nucleic acid sequence in a host cell. For use in the present invention, the transgene product is secreted by the cell to which they are delivered and passes into the blood. Most desirably, such products are soluble or membrane-bound proteins, polypeptides, or peptides. Suitably, any selected molecule which is not secreted by the cell is expressed on the outside of the cell membrane, making it available to the bloodstream. Suitable heterologous molecules, transgenes, and their encoded products are discussed in more detail below.

The inventors have found that by delivering a heterologous molecule encoding a product to the lung via rAAV according to the method of the invention, the product which is secreted from the lung cells or expressed extracellularly is delivered to the bloodstream in sufficient amounts to provide pharmaceutically effective levels of the expressed product in the bloodstream. Thus, the method of the invention provides for administration of rAAV via inhalation in sufficient amounts to transduce lung cells and to provide sufficient transfer levels of transgene (or other heterologous molecules) expression to provide pharmaceutically effective levels of the expressed gene product in the bloodstream. Suitable recombinant AAV constructs are discussed in detail below.

As used herein, lung cells may refer to one or more of the following types of cells: type I pneumocytes, type II pneumocytes, pseudostratified columnar epithelial cells, stratified squamous epithelial cells, gland cells, duct cells, subepithelial connective tissue cells, goblet cells, mucosal cells, submucosal cells, hyaline cartilage cells, perichondrial cells, ciliated columnar cells, basal epithelial cells, brush cells, bronchial epithelial cells, submucosal gland cells, pseudostratified ciliated columnar epithelial cells, lung tissue cells, bronchial respiratory epithelial cells, cuboid epithelial cells of brionchioles, bronchiolar epithelial cells, alveolar cells, squamous (type I) alveolar cells, great (type II) alveolar cells, and alveolar macrophages. “Pharmaceutically effective” levels are levels sufficient to achieve a physiologic effect in a human or veterinary patient, which effect may be therapeutic or immunogenic (e.g., prophylactic).

Dosages of the rAAV will depend primarily on factors such as the condition being treated, the age, weight and health of the patient, and may thus vary among patients. For example, a pharmaceutically effective dose of the rAAV is generally in the range of concentrations of from about 1×10⁵ to 1×10⁵⁰ genomes rAAV, about 10⁸ to 10²⁰ genomes rAAV, about 10¹⁰ to about 10¹⁶ genomes, or about 10¹¹ to 10¹⁶ genomes rAAV. A preferred human dosage may be about 1×10¹³ AAV genomes rAAV. Such concentrations may be delivered in about 0.001 ml to 100 ml, 0.05 to 50 ml, or 10 to 25 ml of a carrier solution.

Conventional pharmaceutically acceptable routes of administration of rAAV may be combined in a regimen which includes delivery by inhalation as described above. These routes include, but are not limited to, direct delivery to the liver, intravenous, intramuscular, subcutaneous, intradermal, oral and other parental routes of administration. Such regimens may involve delivery of the transgene product prior to, or subsequent to, delivery by inhalation according to the present invention.

Optionally, rAAV-mediated delivery according to the invention may be combined with delivery by other viral and non-viral vectors. Such other viral vectors including, without limitation, adenoviral vectors, retroviral vectors, lentiviral vectors. herpes simplex virus (HSV) vectors, and baculovirus vectors may be readily selected and generated according to methods known in the art. Similarly, non-viral vectors, including, without limitation, liposomes, lipid-based vectors, polyplex vectors, molecular conjugates, polyamines and polycation vectors, may be readily selected and generated according to methods known in the art.

When administered by these alternative routes, the dosage is desirable in the range described above. However, the dosage may need to be adjusted to take into consideration an alternative route of administration, or balance the therapeutic benefit against any side effects. Such dosages may vary depending upon the therapeutic application for which the recombinant vector is employed. The levels of expression of the transgene can be monitored to determine the frequency of dosage of viral vectors, preferably AAV vectors, containing the minigene. Optionally, dosage regimens similar to those described for therapeutic purposes may be utilized for non-therapeutic methods, e.g., immunization.

In one embodiment, the method of the invention involves infecting the lung cells of a patient via inhalation of a composition composed of a rAAV containing a selected transgene under the control of sequences which direct expression thereof and AAV5 capsid proteins. As defined herein, AAV5 capsid proteins include hybrid capsid proteins which contain a functional portion of the AAV5 capsid. This embodiment of the invention which uses rAAV with a serotype 5 capsid protein is particularly desirable, because AAV5 capsids are not recognized by neutralizing antibodies to other AAV serotypes. In addition, AAV5 capsids have been found to have tissue tropism for lung cells. However, the methods and compositions of the invention are not limited to rAAV derived from AAV5. One of skill in the art can readily select other rAAV vectors for use in the present invention. These and other suitable rAAV vector constructs are described in more detail below.

II. Pharmaceutical Compositions and Kits

The present invention provides pharmaceutical compositions which are adapted for delivery of a rAAV bearing the selected heterologous molecule to a human or veterinary patient by inhalation.

The rAAV is preferably suspended in a pharmaceutically acceptable delivery vehicle (i.e., physiologically compatible carrier), for administration to a human or non-human mammalian patient. Suitable carriers may be readily selected by one of skill in the art in view of the indication for which the transfer virus is directed. For example, one suitable carrier includes sterile saline, which may be formulated with a variety of buffering solutions (e.g., phosphate buffered saline). Other exemplary carriers include lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin and albumin.

In one embodiment, rAAV of the invention are suitable for applications in which transient transgene expression or delivery of another selected molecule is therapeutic (e.g., p53 gene transfer in cancer and VEGF gene transfer in heart diseases). However, the rAAV are not limited to use where transient transgene expression is desired. The rAAV are useful for a variety of situations in which delivery and expression of a selected molecule is desired. Thus, the compositions of the invention, are useful for any of a variety of delivery applications. Significantly, rAAV having an AAV5 capsid of the invention provide advantages over prior art viruses, in that the rAAV5 of the invention lack serological cross-activity with rAAV of other serotypes and due to tissue tropism for lung.

Suitably, when prepared for use as an inhalant, the pharmaceutical compositions are prepared as fluid unit doses using the rAAV and a suitable pharmaceutical vehicle for delivery by an atomizing spray pump, or by dry powder for insufflation. For use as aerosols, the rAAV can be packaged in a pressurized aerosol container together with a gaseous or liquefied propellant, for example, dichlorodifluormethane, carbon dioxide, nitrogen, propane, and the like, with the usual components such as cosolvents and wetting agents, as may be necessary or desirable.

A pharmaceutical kit of the invention, desirably contains a container for oral or intranasal inhalation, which delivers a metered dose in one, two, or more actuations. Suitably, the kit also contains instructions for use of the spray pump or other delivery device, instructions on dosing, and an insert regarding the active agent (i.e., the transgene and/or rAAV).

A single actuation of a pump spray or inhaler generally delivers contains in the range of about 10⁵ to about 10¹⁵ genome copies (GC), about 10⁸ to about 10¹², and/or about 10¹⁰ GC, in a liquid containing 10 μg to 250 μg carrier, 25 μg to 100 μg, or 40 μg to 50 μg, carrier. Suitably, a dose is delivered in one or two actuations. However, other suitable delivery methods may be readily determined. The doses may be repeated daily, weekly, or monthly, for a predetermined length of time or as prescribed.

III. Recombinant Adeno-Associated Virus

The present invention utilizes recombinant adeno-associated virus (rAAV) in which AAV minigenes are packaged in an AAV capsid.

In one embodiment, the present invention provides AAV minigenes pseudotyped in a capsid of a heterologous AAV serotype, in which either the AAV ITR sequences of the minigene and/or the capsid are of AAV serotype 5 (AAV5).

In another embodiment, the invention provides a rAAV virus, in which both the AAV ITRs and capsid proteins are of the same serotype. In one example, a rAAV containing AAV5 ITRs and an AAV5 capsid, the rAAV contains modified 5′ and/or 3′ ITRs, as described herein. However, the selection of the AAV serotypes for the minigene and/or AAV capsid are not a limitation of the present invention.

As used herein, a “minigene” refers to a construct composed of, at a minimum, AAV ITRs and a heterologous molecule. These components are defined in more detail below. For production of rAAV according to the invention, a minigene may be carried on any suitable vector, including viral vectors, plasmid vectors, and the like.

A “pseudotyped” AAV of the invention refers to a recombinant AAV in which the capsid protein is of a serotype heterologous to the serotype(s) of the ITRs of the minigene. For example, a pseudotyped rAAV may be composed of a minigene carrying AAV5 ITRs and capsid of AAV2, AAV1, AAV3, AAV4, AAV6, or another suitable AAV serotype, where the minigene is packaged in the heterologous capsid. Alternatively, a pseudotyped rAAV may be composed of an AAV5 capsid which has packaged therein a minigene containing ITRs from at least one of the other serotypes. Particularly desirable rAAV composed of AAV5 are described in U.S. patent application Ser. No. 60/200,409, filed Apr. 28, 2000 and International Patent Application No. PCT/USO1/13000, filed Apr. 23, 2001, both of which are incorporated by reference herein.

The AAV sequences used in generating the minigenes, vectors, and capsids, and other constructs used in the present invention may be obtained from a variety of sources. For example, the sequences may be provided by AAV type 5, AAV type 2, AAV type 1, AAV type 3, AAV type 4, AAV type 6, or other AAV serotypes or other densoviruses. A variety of these viral serotypes and strains are available from the American Type Culture Collection, Manassas, Virginia, or are available from a variety of academic or commercial sources. Alternatively, it may be desirable to synthesize sequences used in preparing the vectors and viruses of the invention using known techniques, which may utilize AAV sequences which are published and/or available from a variety of databases. The source of the sequences utilized in preparation of the constructs of the invention is not a limitation of the present invention.

A. AAV Minigene

The AAV minigene contains, at a minimum, AAV inverted terminal repeat sequences (ITRs) and a heterologous molecule for delivery to a host cell. Most suitably, the minigene contains AAV 5′ ITRs and 3′ ITRs located 5′ and 3′ to the heterologous molecule, respectively. However, in certain embodiments, it may be desirable for the minigene to contain the 5′ ITR and 3′ ITR sequences arranged in tandem, e.g., 5′ to 3′ or head-to-tail, or in yet another configuration. In still other embodiments, it may be desirable for the minigene to contain multiple copies of the ITRs, or to have 5′ ITRs (or conversely, 3′ ITRs) located both 5′ and 3′ to the heterologous molecule. The ITR sequences may be located immediately upstream and/or downstream of the heterologous molecule, or there may be intervening sequences. The ITRs may be selected from AAV5, or from among the other AAV serotypes, as described herein. The heterologous molecule may be any substance which is desired to be delivered to a cell, including, without limitation, a polypeptide, protein, enzyme, carbohydrate, chemical moiety, or nucleic acid sequence which may include oligonucleotides, RNA, and/or DNA.

In one embodiment, the heterologous molecule may be a nucleic acid molecule which introduces specific genetic modifications into human chromosomes, e.g., for correction of mutated genes. See, e.g., D. W. Russell & R. K. Hirata, NATURE GENETICS, 18:325-330 (April 1998). In another desirable embodiment, the heterologous molecule is a transgene, as defined herein. Selection of the heterologous molecule delivered by the AAV minigene is not a limitation of the present invention.

1. ITR Sequences

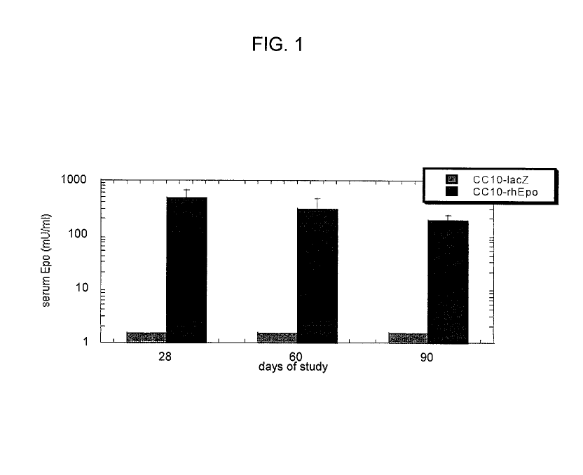

As defined herein, an “AAV5” minigene contains ITRs of AAV serotype 5. (These sequences are illustrated, in FIG. 1 of J. A. Chiorini et al, J. VIROL, 73(2):1309-1319 (Feb. 1999), and are available from GenBank under accession no. AF085716) . Preferably, substantially the entire ITR sequences are used in the molecule, although some degree of modification of these sequences is permissible. For example, the inventors have found that it is possible to utilize a 175-bp 5′ ITR (13 bp deleted at the 3′ end of the 5′ ITR) and an 182-bp 3′ ITR (6 bp at the 5′ end of the 3′ ITR), whereas the art has described 188 bp 5′ and 3′ ITRs (Chiorini, cited above). The ability to modify these ITRs sequences is within the skill of the art.

Minigenes containing ITRs from other AAV serotypes are defined similarly. For example, an “AAV2” minigene contains AAV2 ITRs. These ITR sequences are about 145 bp in length. (See, e.g., Chiorini, cited above; also, see, B. J. Carter, in “Handbook of Parvoviruses”, e.g., P. Tijsser, CRC Press, pp. 155-168 (1990)). However, the present invention does not require that the minigene contain both 5′ and 3′ ITRs from a single serotype source. Optionally, a minigene may contain 5′ ITRs from one serotype and 3′ ITRs from a second serotype. For ITRs from any selected AAV serotype, as with the AAV5 ITRs, the entire ITR sequences may be used in the minigene, or minor modifications may be made to the sequences.

2. Transgene

In one embodiment, the heterologous molecule of the AAV minigene comprises a transgene. As described above, for use in the present invention, the transgene product is preferably a soluble or membrane-bound protein, polypeptide, peptide, enzyme, or other molecule.

The composition of the transgene will depend upon the use to which the rAAV of the invention will be put. For example, one type of nucleic acid sequence which may be included in a transgene includes a reporter sequence, which upon expression produces a detectable signal. Such reporter sequences include without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These sequences, when associated with regulatory elements which drive their expression, provide signals detectable by conventional means, including enzymatic, radiographic, calorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of virus is detected by assays for beta-galactosidase activity. Where the transgene is luciferase, the virus may be measured by light production in a luminometer.

Optionally such reporter sequences, even when non-secretable, may be used in conduction with a construct containing a second transgene. In such embodiments, the presence of the reporter sequences may be used to detect transfection levels of the targeted host cells.

Desirably, the present invention utilizes a transgene which comprises a non-marker sequence encoding a product which is useful in biology and medicine, such as proteins, peptides, anti-sense nucleic acids (e.g., RNAs), enzymes, or catalytic RNAs.

The encoded product may be used to achieve a physiologic effect in a patient, e.g., therapeutic, or immunogenic (e.g., to provide passive immunity or to stimulate a cellular and/or humoral immune response). For example, therapeutic molecules may be used to correct or ameliorate gene deficiencies, such as deficiencies in which normal genes are expressed at less than normal levels or deficiencies in which the functional gene product is not expressed.

The invention further includes using multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. In another embodiment, different subunits of a protein may be encoded by the same transgene.

However, the selected transgene may encode any product desirable for study. The selection of the transgene sequence is not a limitation of this invention.

Other useful products which may be encoded by the transgene include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, endostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1- 15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog, tyrosine hydroxylase, and soluble decoy receptors such as FLT-1.

Other useful transgene products include proteins that regulate the immune system including, without limitation, cytokines and lymphokines such as thrombopoietin (TPO), interleukins (IL) IL-1 through IL-18, monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand.

Gene products produced by the immune system are also useful in the invention. These include, without limitations, immunoglobulins IgG, IgM, IgA, IgD and IgE, chimeric immunoglobulins, humanized antibodies, single chain antibodies, antibody fragments which retain the binding specificity and ability of their parent antibody (i.e., functional fragments), T cell receptors, chimeric T cell receptors, single chain T cell receptors, class I and class II MHC molecules, as well as monoclonal antibodies, engineered antibodies and immunoglobulins and MHC molecules. Particularly desirable antibodies and functional fragments thereof include those which target soluble proteins, membrane-bound proteins, oncogene products, and viral proteins, among others. For example, one suitable antibody (or functional fragment thereof or other secreted protein) has high affinity for presinillin. For example, such an antibody may include an anti-presenillin single chain antibody which may be useful for treatment of Alzheimer's Disease. Also useful may be a synthetic zinc finger transcription factor that dominantly represses the presinillin promoter. Other useful gene products also include regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The transgene may also contain genes encoding products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

Other useful gene products include, non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a gene. Other suitable products may be readily selected by one of skill in the art. The selection of the product encoded by the transgene is not considered to be a limitation of this invention.

3. Regulatory Elements

The transgene includes appropriate sequences that are operably linked to the nucleic acid sequences encoding the product of interest to promote its expression in a host cell. “Operably linked” sequences include both expression control sequences that are contiguous with the coding sequences for the product of interest and expression control sequences that act in trans or at a distance to control the expression of the product of interest. In addition to being useful in the transgene, the regulatory elements described herein may also be used in other heterologous molecules and the other constructs described in this application.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein processing and/or secretion. A great number of expression control sequences, e.g., native, constitutive, inducible and/or tissue-specific, are known in the art and may be utilized to drive expression of the gene, depending upon the type of expression desired. For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence. In one embodiment, the bovine growth hormone polyA used.

In one embodiment, lung-specific promoters are desired. Examples of such lung-specific promoters include Clara cell secretory protein (CCSP) promoter (RM Graham, et al, AM J RESPIR CRIT CARE MED, 164(2): 307-313 (Jul. 15 2001)); the lung-specific surfactant protein C promoter (A. Ehrhardt, et al, BR J CANCER, 84(6):813-818 (Mar 23, 2001)); Jaagskiekte sheep retrovirus (JSRV) long terminal repeat (M. Palmarini, et al, J. VIROL., 74(13):5776-5787 (July 2000)); rat aquaporin-5 promoter (Z. Borok, et al, J BIOL CHEM., 275(34):26507-26514 (Aug 25 2000)). Still other lung-specific promoters may be readily selected by one of skill in the art for use in the invention. Alternatively, non-tissue-specific promoters may be readily selected.

In another embodiment, high-level constitutive expression desired. Examples of such promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, CELL, 41:521-530 (1985)), the SV40 promoter, the dihydrofolate reductase promoter, the β-actin promoter, the phosphoglycerol kinase (PGK) promoter, and the EF1α promoter (Invitrogen). Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, PROC. NATL. ACAD. Sci. USA, 93:3346-3351 (1996)), the tetracycline-repressible system (Gossen et al, PROC. NATL. ACAD. SCI. USA, 89:5547-5551 (1992)), the tetracycline-inducible system (Gossen et al, SCIENCE, 268:1766-1769 (1995); see also Harvey et al, CURR. OPIN. CHEM. BIOL., 2:512-518 (1998)), the RU486-inducible system (Wang et al, NAT. BIOTECH., 15:239-243 (1997) and Wang et al, GENE THER., 4:432-441 (1997)) and the rapamycin-inducible system (Magari et al, J. CLIN. INVEST., 100:2865-2872 (1997)). Other types of inducible promoters which may be useful in the transgenes and other constructs described herein are those which are regulated by a specific physiological state, e.g., temperature, acute phase, a particular differentiation state of the cell, or in replicating cells only.

In another embodiment, the native promoter for the selected gene product will be used. The native promoter may be preferred when it is desired that expression of the product should mimic the native expression. The native promoter may be used when expression of the product must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

The regulatory sequences useful in the constructs of the present invention may also contain an intron, desirably located between the promoter/enhancer sequence and the gene. One possible intron sequence is also derived from SV-40, and is referred to as the SV-40 T intron sequence. In certain cases, e.g., where a single transgene includes the DNA encoding each of the subunits, it may be desirable to separate the DNA for each subunit by an internal ribozyme entry site (IRES). This is desirable when the size of the DNA encoding each of the subunits is small, e.g., total of the DNA encoding the subunits and the IRES is less than five kilobases. Alternatively, other methods which do not require the use of an IRES may be used for co-expression of proteins. For example, as alternative to an IRES, the DNA may be separated by sequences encoding a 2A peptide, which self-cleaves in a post-translational event. See, e.g., M. L. Donnelly, et al, J. GEN. VIROL., 78(Pt 1):13-21 (Jan 1997); S. Furler et al, GENE THER., 8(11):864-873 (June 2001); H. Klump, et al., GENE THER., 8(10):811-817 (May 2001). Another suitable sequence includes the woodchuck hepatitis virus post-transcriptional element. (See, e.g., L. Wang and I. Verma, PROC. NATL. ACAD. SCI., (1999)). Still other methods may involve the use of a second internal promoter, an alternative splice signal, another co- or post-translational proteolytic cleavage strategy, among others which are known to those of skill in the art. Selection of these and other common vector and regulatory elements are conventional and many such sequences are available. See, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989.

One of skill in the art may make a selection among these expression control sequences without departing from the scope of this invention. Suitable promoter/enhancer sequences may be selected by one of skill in the art using the guidance provided by this application. Such selection is a routine matter and is not a limitation of the transgene or other construct. For instance, one may select one or more expression control sequences operably linked to the coding sequence of interest for use in a transgene for insertion in a “minigene” which is composed of the 5′ ITRs, a transgene, and 3′ ITRs. Such a minigene may have a size in the range of several hundred base pairs up to about 30 kb. Thus, this system permits a great deal of latitude in the selection of the various components of the minigene, particularly the selected transgene, with regard to size. Provided with the teachings of this invention, the design of such a minigene can be made by resort to conventional techniques.

After following one of the methods for packaging the minigene taught in this specification, or as taught in the art, one may infect suitable cells in vitro or in vivo. Where the heterologous molecule comprises a transgene, the number of copies of the transgene in the cell may be monitored by Southern blotting or quantitative polymerase chain reaction (PCR). The level of RNA expression may be monitored by Northern blotting or quantitative reverse transcriptase (RT)-PCR. The level of protein expression may be monitored by Western blotting, immunohistochemistry, ELISA, RIA, or tests of the transgene's encoded product's biological activity. Thus, one may easily assay whether a particular expression control sequence is suitable for a specific transgene, and choose the expression control sequence most appropriate for expression of the desired transgene. Suitable methods for detecting the presence of other heterologous molecules delivered via the rAAV of the invention are known to those of skill in the art and are not a limitation of the present invention.

B. AAV Capsid

In a one embodiment, the present invention provides a pseudotyped rAAV in which a non-AAV5 minigene is packaged in an AAV5 capsid or an AAV5 transfer vector is packaged in a non-AAV5 capsid. Suitably, the sequences providing the AAV capsid protein of the selected serotype may be obtained from any suitable source, as with the other AAV sequences described herein.

In another embodiment, the invention provides a rAAV virus, in which both the AAV ITRs and capsid protein are of serotype 5. In this embodiment, the virus preferably contains modified 5′ and/or 3′ ITRs. More particularly, the virus desirably contains a 175-bp 5′ ITR and a 182-bp 3′ ITR. Desirably, in this embodiment, the rAAV5 virus further contains a promoter and an intron upstream of the transgene, and a woodchuck hepatitis virus post-transcriptional element and a bovine growth hormone polyA signal downstream of the transgene.

In still another embodiment, the invention provides a rAAV virus, in which both the AAV ITRs and capsid protein are independently selected from among AAV serotypes, including, without limitation, AAV1, AAV2, AAV3, AAV4, AAV5, and AAV6. For example, the invention may utilize an rAAV1 vector, a rAAV2 vector, an rAAV2/1 vector, and rAAV1/2 vector and/or an rAAV2/5 vector, as desired. By way of example and without limitation, other suitable rAAV vectors may be derived from the following combinations:

| ITRs | Rep | Cap |
|---|---|---|
| 1 | 1 | 1 |
| 1 | 2 | 2 |
| 2 | 2 | 1 |
| 2 | 2 | 2 |
| 2 | 5 | 5 |
| 5 | 5 | 5 |

As defined herein, AAV capsid proteins include hybrid capsid proteins which contain a functional portion of one or more AAV capsid proteins. Such hybrid capsid proteins may be constructed such that a fragment of a capsid derived from one serotype is fused to a fragment of a capsid from another serotype to form a single hybrid capsid which is useful for packaging of an AAV minigene.

The rAAV of the invention, composed of an AAV transfer vector packaged in an AAV capsid described herein, may be produced utilizing the following methods or other suitable methods known in the art.

IV. Production of rAAV

The present invention provides a method which permits the production of a pseudotyped AAV virus, in which an AAV5 minigene is packaged in a heterologous AAV serotype capsid or in which a non-AAV5 serotype minigene is packaged in an AAV5 capsid. The inventors have found that this pseudotyping can be achieved by utilizing a rep protein (or a functional portion thereof) of the same serotype or a cross-reactive serotype as that of the ITRs found in the minigene in the presence of sufficient helper functions to permit packaging. Thus, an AAV2 minigene can be pseudotyped in an AAV5 capsid by use of a rep protein from AAV2 or a cross-reactive serotype, e.g., AAV1, AAV3, AAV4 or AAV6. Similarly, an AAV minigene containing AAV1 5′ ITRs and AAV2 3′ ITRs may be pseudotyped in an AAV5 capsid by use of a rep protein from AAV 1, AAV2, or another cross-reactive serotype. However, because AAV5 is not cross-reactive with the other AAV serotypes, an AAV5 minigene can be pseudotyped in a heterologous AAV capsid only by use of an AAV5 rep protein.

Thus, in one embodiment, the invention provides a method of pseudotyping an AAV minigene in an AAV serotype 5 capsid. The method involves culturing in a host cell an AAV minigene containing ITRs which are derived from one or more serotypes heterologous to AAV5, a nucleic acid sequence driving expression of the AAV5 capsid protein, and a functional portion of an AAV rep of the same (or a cross-reactive) serotype as that of the AAV ITRs, in the presence of sufficient helper functions to permit packaging of the minigene in the AAV5 capsid.

In another embodiment, the invention provides a method of pseudotyping an AAV5 minigene in an AAV capsid from another serotype. The method involves culturing in a host cell an AAV minigene containing AAV5 ITRs, a nucleic acid sequence driving expression of the AAV capsid protein, and a functional portion of an AAV5 rep, in the presence of sufficient helper functions to permit packaging of the AAV ITR-heterologous molecule-AAV ITR minigene in the AAV capsid.

In still another embodiment, the invention provides a helper virus-free method of producing rAAV5 virus, in which both the AAV ITRs and capsid proteins are of serotype 5. In this embodiment, the virus preferably contains modified 5′ and/or 3′ ITRs.

In a further embodiment, the rAAV may be produced by convention methods using AAV ITRs and capsid proteins selected from among available AAV serotypes.

In yet a further embodiment, the rAAV of the invention may be produced by in vitro packaging. In this embodiment, the capsid proteins are produced in host cells and extracted from the host cells, using production and purification techniques similar to those described for packaging of the rAAV in host cells. The extracted capsid proteins are then utilized for in vitro packaging of the virus. Suitable techniques for in vitro packaging are known to those of skill in the art. See, e.g., X. Zhou and N. Muzyczka, J. VIROL, 72:3341-3347 (Apr. 1998). Selection of the appropriate packaging method for the rAAV of the invention is not a limitation of the present invention.

A. Delivery of Required Components to Packaging Host Cell

The components required to be cultured in the host cell to package the AAV minigene in the AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the invention may be delivered to the packaging host cell in the form of any genetic element, e.g., naked DNA, a plasmid, phage, transposon, cosmid, virus, etc. which transfer the sequences carried thereon. The selected genetic element may be delivered by any suitable method, including transfection, electroporation, liposome delivery, membrane fusion techniques, high velocity DNA-coated pellets, viral infection and protoplast fusion.

The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

1. Delivery of Minigene

Currently, the minigene is preferably carried on a plasmid which is delivered to a host cell by transfection. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ITR) contain sequences permitting replication of the minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the minigene is transfected into the cell, where it may exist transiently or preferably as an episome. Alternatively, the minigene (carrying the 5′ AAV ITR-heterologous molecule-3′ ITR) may be stably integrated into a chromosome of the host cell. Suitable transfection techniques are known and may readily be utilized to deliver the minigene to the host cell.

Generally, when delivering the vector comprising the minigene by transfection, the vector is delivered in an amount from about 5 μg to about 100 μg DNA, and preferably about 10 to about 50 μg DNA to about 1×10⁴ cells to about 1×10¹³ cells, and preferably about 10⁵ cells. However, the relative amounts of vector DNA to host cells may be adjusted, taking into consideration such factors as the selected vector, the delivery method and the host cells selected.

2. Rep and Cap Sequences

In addition to the minigene, the host cell must also contain the sequences which drive expression of the capsid protein of the selected AAV serotype in the host cell and rep sequences of the same serotype as the serotype of the AAV ITRs found in the minigene. The AAV cap and rep sequences may be independently obtained from an AAV source as described above and may be introduced into the host cell in any manner known to one in the art as described above. Additionally, when pseudotyping an AAV vector in an AAV5 capsid, the sequences encoding each of the essential rep proteins may be supplied by the same AAV serotype, or the sequences encoding the rep proteins may be supplied by different, but cross-reactive, AAV serotypes (e.g., AAV1, AAV2, AAV3, AAV4 and AAV6). For example, the rep78/68 sequences may be from AAV2, whereas the rep52/40 sequences may from AAV1.

In one embodiment, the host cell stably contains the capsid protein under the control of a suitable promoter, such as those described above. Most desirably, in this embodiment, the capsid protein is expressed under the control of an inducible promoter. In another embodiment, the capsid protein is supplied to the host cell in trans. When delivered to the host cell in trans, the capsid protein may be delivered via a plasmid which contains the sequences necessary to direct expression of the selected capsid protein in the host cell. Most desirably, when delivered to the host cell in trans, the plasmid carrying the capsid protein also carries other sequences required for packaging the rAAV, e.g., the rep sequences.

In another embodiment, the host cell stably contains the rep sequences under the control of a suitable promoter, such as those described above. Most desirably, in this embodiment, the essential rep proteins are expressed under the control of an inducible promoter. In another embodiment, the rep proteins are supplied to the host cell in trans. When delivered to the host cell in trans, the rep proteins may be delivered via a plasmid which contains the sequences necessary to direct expression of the selected rep proteins in the host cell. Most desirably, when delivered to the host cell in trans, the plasmid carrying the capsid protein also carries other sequences required for packaging the rAAV, e.g., the rep and cap sequences.

Thus, in one embodiment, the rep and cap sequences may be transfected into the host cell on a single nucleic acid molecule and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV construct comprising the minigene. The vector may comprise one or more of the genes encoding the helper functions, e.g., the adenoviral proteins E1, E2a, and E40RF6, and the gene for VAI RNA.

Preferably, the promoter used in this construct may be any of the constitutive, inducible or native promoters known to one of skill in the art or as discussed above. In one embodiment, an AAV P5 promoter sequence is employed. The selection of the AAV to provide any of these sequences does not limit the invention.

In another preferred embodiment, the promoter for rep is an inducible promoter, as are discussed above in connection with the transgene regulatory elements. One preferred promoter for rep expression is the T7 promoter. The vector comprising the rep gene regulated by the T7 promoter and the cap gene, is transfected or transformed into a cell which either constitutively or inducibly expresses the T7 polymerase. See WO 98/10088, published Mar. 12, 1998.

The spacer is an optional element in the design of the vector. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the X phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employed for constructing embodiments of this invention are conventional genetic engineering or recombinant engineering techniques such as those described in the references above. While this specification provides illustrative examples of specific constructs, using the information provided herein, one of skill in the art may select and design other suitable constructs, using a choice of spacers, P5 promoters, and other elements, including at least one translational start and stop signal, and the optional addition of polyadenylation sites.

In another embodiment of this invention, the rep or cap protein may be provided stably by a host cell.

3. The Helper Functions

The packaging host cell also requires helper functions in order to package the rAAV of the invention. Optionally, these functions may be supplied by a herpesvirus. Most desirably, the necessary helper functions are provided from an adenovirus source. In one currently preferred embodiment, the host cell is provided with and/or contains an E1a gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell.

The DNA sequences encoding the adenovirus E4 ORF6 genes and the E1 genes and/or E2a genes useful in this invention may be selected from among any known adenovirus type, including the presently identified 46 human types [see, e.g., Horwitz, cited above and American Type Culture Collection]. Similarly, adenoviruses known to infect other animals may supply the gene sequences. The selection of the adenovirus type for each E1, E2a, and E4 ORF6 gene sequence does not limit this invention. The sequences for a number of adenovirus serotypes, including that of serotype Ad5, are available from Genbank. A variety of adenovirus strains are available from the American Type Culture Collection (ATCC), Manassas, Va., or are available by request from a variety of commercial and institutional sources. Any one or more of human adenoviruses Types 1 to 46 may supply any of the adenoviral sequences, including E1, E2a, and/or E4 ORF6.

By “adenoviral DNA which expresses the E1a gene product”, it is meant any adenovirus sequence encoding E1a or any functional E1a portion. Adenoviral DNA which expresses the E2a gene product and adenoviral DNA which expresses the E4 ORF6 gene products are defined similarly. Also included are any alleles or other modifications of the adenoviral gene or functional portion thereof. Such modifications may be deliberately introduced by resort to conventional genetic engineering or mutagenic techniques to enhance the adenoviral function in some manner, as well as naturally occurring allelic variants thereof. Such modifications and methods for manipulating DNA to achieve these adenovirus gene functions are known to those of skill in the art.

The adenovirus E1a, E1b, E2a, and/or E40RF6 gene products, as well as any other desired helper functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example.

B. Host Cells And Packaging Cell Lines

The host cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA. In a preferred embodiment, the host cell is one that has rep and cap stably transfected in the cell.

One host cell useful in the present invention is a host cell stably transformed with the sequences encoding rep and cap, and which is transfected with the adenovirus E1, E2a, and E40RF6 DNA and a construct carrying the minigene as described above. Stable rep and/or cap expressing cell lines, such as B-50 (PCT/US98/19463), or those described in U.S. Pat. No. 5,658,785, may also be similarly employed. Another desirable host cell contains the minimum adenoviral DNA which is sufficient to express E4 ORF6.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins).

Thus produced, the rAAV may be used to prepare the compositions and kits described herein, and used in the method of the invention.

For example, in one embodiment, the invention involves infecting the lung cells of a patient via inhalation of a composition composed of a rAAV containing a selected transgene under the control of sequences which direct expression thereof and AAV5 capsid proteins. The use of rAAV derived from AAV5 capsids is particularly desirable, as they allow for long-term gene expression as compared to other vectors which transduce lung cells efficiently (e.g., adenoviral vectors). Additionally, rAAV having capsids derived from AAV5 or a fragment thereof transduce lung cells in a manner which allows for secretion of proteins into the blood stream (in contrast to adenoviral vectors which secrete into the lumen of the lung rather than into the blood stream).

In another embodiment, the invention provides a method of infecting a selected host cell with a rAAV containing a AAV5 transfer vector packaged in a capsid protein of another AAV serotype by inhalation. Optionally, a sample from the host may be first assayed for the presence of antibodies to a selected AAV serotype. A variety of assay formats for detecting neutralizing antibodies are well known to those of skill in the art. The selection of such an assay is not a limitation of the present invention. See, e.g., Fisher et al, NATURE MED., 3(3):306-312 (March 1997) and W. C. Manning et al, HUMAN GENE THERAPY, 9:477-485 (March 1, 1998). The results of this assay may be used to determine from which serotype the capsid protein will be preferred for delivery, e.g., by the absence of neutralizing antibodies specific for that capsid serotype.

In another embodiment of this method, the delivery of vector with an AAV5 capsid protein may precede or follow delivery of a heterologous molecule (e.g., gene) via a vector with a different serotype AAV capsid protein. Thus, delivery via multiple rAAV vectors may be used for repeat delivery of a desired molecule to a selected host cell. Desirably, subsequently administered rAAV carry the same minigene as the first rAAV vector, but the subsequently administered vectors contain capsid proteins of serotypes which differ from the first vector. For example, if a first rAAV has an AAV5 capsid protein, subsequently administered rAAV may have capsid proteins selected from among the other serotypes, including AAV2, AAV1, AAV3A, AAV3B, AAV4 and AAV6. Alternatively, if a first rAAV has an AAV2 capsid protein, subsequently administered rAAV may have an AAV5 capsid. Still other suitable combinations will be readily apparent to one of skill in the art.

The following examples illustrate production of exemplary rAAV and several other aspects and embodiments of the invention. These examples are not limiting.

### EXAMPLE 1

### Pseudotyping of AAV2 Transfer Vector in AAV Capsid

A. pAAV2.1 lacZ

The AAV2 plasmid which contains the AAV2 ITRs and the beta-galactosidase gene of E. coli with the cytomegalovirus (CMV) promoter was constructed as described below. Plasmid pAAV2.1 lacZ contains 6 elements:

(i) Plasmid Backbone pAAV2.1 Containing the AAV2 ITRs:

A pUC-19 based expression plasmid (pZAC3.1) was digested with the restriction enzymes BglII and ClaI and the cohesive ends filled in using Pfu Polymerase (Stratagene). Afterwards, an EcoRi linker (New England Biolabs) was introduced. After EcoRI digestion, the construct was religated, resulting in plasmid pAAV2.1, which provides the plasmid backbone containing the AAV2 5′ ITRs and AAV2 3′ ITRs.

(ii) CMV Promoter:

The CMV promoter was amplified with Pfu Polymerase with pEGFP-C1 (Clontech) as template using primers:

CLONE/CMV promoter/NheI+:
SEQ ID NO:1: AAGCTAGCTAGTTATTAATAGTAATC
CLONE/CMV promoter/PstI−:
SEQ ID NO:2: AACTGCAGGATCTGACGGTTCACTAAAC

and ligated into pCR4topo (Invitrogen). The CMV promoter fragment was cut out with EcoRI and PstI, so that an EcoRI site flanks the NheI site.

(iii) Chimeric intron:

The chimeric intron was amplified with Pfu Polymerase with pCI (Promega) as template using primers:

CLONE/SV40 intronPst+:
SEQ ID NO:3: AACTGCAGAAGTTGGTCGTGAGGCAC
CLONE/SV40 intron/NotI−:
SEQ ID NO:4: AAGCGGCCGCCTGGACACCTGTGGAGAAAG

and afterwards digested with the restriction enzymes PstI and NotI, resulting in the chimeric intron fragment.

(iv) Beta-galactosidase Coding Sequence:

The beta-galactosidase coding sequence was amplified with Pfu Polymerase (Stratagene) with E. coli genomic DNA (ATCC) as template using primers:

CLONE/lacZ/NotI30 :
SEQ ID NO:5: AAGCGGCCGCCATGACCATGATTACGGATTC
CLONE/lacZ/BamHI−:
SEQ ID NO:6: TTGGATCCTTATTTTTGACACCAGAC

and afterwards digested with the restriction enzymes NotI and BamHI resulting in the beta-galactosidase fragment.

(v) Woodchuck Hepatitis Post-Regulatory Element (WPRE):

The WPRE element was amplified with Pfu Polymerase with woodchuck hepatitis virus DNA (ATCC) as template using primers:

CLONE/WPRE/BamHI+:
SEQ ID NO:7: AAGGATCCAATCAACCTCTGGATTAC
CLONE/WRPRE/BglII−:
SEQ ID NO:8: TTAGATCTCGAAGACGCGGAAGAGGCCG

and afterwards digested with the restriction enzymes BamHI and BgII resulting in the WPRE fragment.

(vi) Bovine growth hormone polyadenylation signal:

The bovine growth hormone polyadenylation signal (BGHpA) was amplified with Pfu Polymerase with pCDNA3. 1 (Invitrogen) as template using primers:

CLONE/BOGH pA/BgIII+:
SEQ ID NO:9: TTTAGATCTGCCTCGACTGTGCCTTCTAG
CLONE/BGH pA/XhoI−:
SEQ ID NO:10: AACTCGAGTCCCCAGCATGCCTGCTATTG

and ligated into pCR4 topo (Invitrogen). The BGHpA fragment was excised with BglII and EcoRI so that EcoRI flanks the XhoI site.

In order to assemble pAAV2.1 lacZ, the plasmid pAAV2.1 was cut with EcoRI and ligated together with the CMV promoter fragment (EcoRI/PstI), chimeric intron fragment (PstI/NotI), beta-galactosidase coding sequence (NotI/BamHI), WPRE element (BamHI/BglII), BGHpA fragment (BglII/EcoRI) in a multi-fragment ligation resulting in plasmid pAAV2.1 lacZ.

B. Cloning of p600 Trans

The P5 promoter was excised from pCR-p5 by BamHI and XhoI, filled in by Klenow and then cloned into pMMTV-Trans at SmaI+ClaI to obtain pP5-X-Trans. The construction of pCR-p5 and pMMTV-Trans were described previously (Xiao et al, J. VIROL, 73:3994-4003 (1999)). There is a unique EcoRV site between the P5 promoter and the initiation codon of Rep78 in pP5-X-Trans. All helper plasmids are made by cloning either the 100 bp ladder or 500 bp ladder from Gibco BRL using the EcoRV site in p5-X-Trans. These series of plasmids are designated as pSY, where Y indicates the size of the spacer which ranges from 100 bp to 5 kb. Thus, the p600trans plasmid contains a 600 bp insert consisting of the 500 bp ladder and a 100 bp spacer.

Plasmid p600trans (containing the rep and cap proteins of AAV serotype 2) was subjected to PCR amplification with Pfu Polymerase (Stratagene) according to manufacturer's instructions (Seemless cloning kit) using primers:

Clone/AAV2rep.cap seemless+:
SEQ ID NO:11: AGTTACTCTTCTTGCTTGTTAATCAATAAACCGTTTAATTCG
Clone/AAV2rep.cap seemless−:
SEQ ID NO:12: AGTTACTCTTCACCTGATTTAAATCATTTATTGTTCAAAGATGC

Following PCR, the plasmid was digested with restriction enzyme Eam 1104 I, to provide fragment p600 trans ΔCAP-ORF. This fragment contains the sequences encoding AAV2 rep proteins 78 and 52. The AAV2 Rep68 and Rep40 proteins have a amino acid deletion as compared to the wt AAV Rep68 and Rep 40. In addition the last five C-terminal amino acids are substituted (i.e., sAA, sAA, sAA, sAA, dAA, dAA, with s: substituted, d: deleted), because of the overlap between their C-termini and the AAV5 VP1 open reading frame.

AAV5-CAP-ORF (U. Bantel-Schaal, J. VIROL., 73/2:939-947 (Feb. 1999)) was amplified by PCR with Pfu Polymerase as above, using the primers:

Clone/AAV5 Cap seemless+:
SEQ ID NO: 13: AGTTACTCTTCCAGGTATGTCTTTTGTTGATCACCCTCCAGATTGG
Clone/AAV5 CAP seemless−:
SEQ ID NO:14: AGTTACTCTTCAGCAATTAAAGGGGTCGGGTAAGGTATCGGGTTC

and thereafter digested with Eam 1104I, to provide AAV5-CAP5. This fragment contains the sequences encoding the AAV5 capsid protein.

The fragments resulting from the above described PCR amplifications, p600 trans ΔCAP-ORF and AAV5-CAP5, were ligated according to manufacturer's instructions. The resulting plasmid contains the AAV2 rep sequences for Rep78/68 under the control of the AAV2 P5 promoter, and the AAV2 rep sequences for Rep52/40 under the control of the AAV2 P19 promoter. The AAV5 capsid sequences are under the control of the AAV2 P40 promoter, which is located within the Rep sequences. This plasmid further contains a spacer 5′ of the rep ORF.

C. Production of Pseudotyped rAAV

The rAAV particles (AAV2 vector in AAV5 capsid) were generated using an adenovirus-free method. Briefly, the cis plasmid (pAAV2.1 lacZ plasmid containing AAV2 ITRs), and the trans plasmid pAdΔF6 (containing the AAV2 rep and AAV5 cap) and a helper plasmid, respectively, were simultaneously co-transfected into 293 cells in a ratio of 1:1:2 by calcium phosphate precipitation.

For the construction of the pAd helper plasmids, pBG10 plasmid was purchased from Microbix (Canada). A RsrII fragment containing L2 and L3 was deleted from pBHG10, resulting in the first helper plasmid, pAdΔF13. Plasmid AdΔF1 was constructed by cloning Asp700/SalI fragment with a PmeI/Sgf1 deletion, isolating from pBHG10, into Bluescript. MLP, L2, L2 and L3 were deleted in the pAdΔF1. Further deletions of a 2.3 kb NruI fragment and, subsequently, a 0.5 kb RsrII/Nru1 fragment generated helper plasmids pAdΔF5 and pAdΔF6, respectively. The helper plasmid, termed pΔF6, provides the essential helper functions of E2a and E4 ORF6 not provided by the E1-expressing helper cell, but is deleted of adenoviral capsid proteins and functional E1 regions).

Typically, 50 μg of DNA (cis:trans:helper) was transfected onto a 15 cm tissue culture dish. The 293 cells were harvested 72 hours post-transfection, sonicated and treated with 0.5% sodium deoxycholate (37° C. for 10 min.) Cell lysates were then subjected to two rounds of a CsCl gradient. Peak fractions containing rAAV vector are collected, pooled and dialyzed against PBS.

### EXAMPLE 2

### Production of rAAV5

A. pAA V5.9 LacZ

The AAV5 plasmid which contains the modified AAV5 ITRs and the nucleus-localized beta-galactosidase gene with a cytomegalovirus (CMV) promoter was constructed as described below.

The plasmid, pAAVRnLacZ (J. A. Chiorini et al, HUM. GENE THER., 6:1531-1541 (1995)), was subjected to PCR amplification with Pfu Polymerase (Stratagene), according to manufacturer's instructions using the primers shown below, to provide plasmid pAAV5.1.

Clone/AAV5/NheI-XhoI:
SEQ ID NO:15: AAACTCGAGATTGCTAGCTCACTGCTTACAAAACCCCCTTGCTTGAG
Clone/AAV5/XhoI+:
SEQ ID NO: 16: TTCACAGCTTACAACATCTACAAAAC

pAAV5.1 was digested with pAAV5.1 with restriction enzymes NheI and XhoI and the NheI/XhoI fragment containing the lacZ expression cassette of pAAV2.1lacZ (described above) was inserted, resulting in the plasmid pAAV5.1acZ

B. Construction ofpAAV5.1eGFP

eGFP was amplified with Pfu Polymerase (Stratagene) according to manufacturer's instructions using pEGFP-C 1 (Clontech) as template with the primers:

CLONE/eGFP/NotI+:
SEQ ID NO:17: AAAGCGGCCGCCATGGTGAGCAAGGGCGAGGAG
CLONE/eGFP/HindIII-BamHI−:
SEQ ID NO:18: AAGGATCCAAGCTTATTACTTGTACAGCTCGTCCATGCC

and digested with the restriction enzymes NotI and BamHI resulting in the fragment eGFP. This fragment was ligated into pAAV5.1lacZ in which the lacZ coding sequence was removed by digestion with NotI and BamHI resulting in the plasmid pAAV5.1 eGFP.

C. Transduction Efficiency of rAAV with AAV5 Capsids

These rAAV were injected (1×10¹⁰ to 4×10¹⁰ genomes) into murine lung, liver, intestine and muscle tissue with recombinant AAV2 as a control vector. Preliminary results suggest a higher transduction efficiency of lung, intestine and muscle tissue by AAV5 than by AAV2. This indicates that vectors containing AAV5 capsids are extremely useful for targeting lungs, e.g., for the correction of the autosomal recessive inherited disease Cystic Fibrosis (CF) by delivery of the CFTR gene, and muscle.

### EXAMPLE 3

### In vivo Delivery of Therapeutic Proteins by AAV2/5 Vectors

In the present study, it was demonstrated that aerosolized AAV2/5 encoding a secretable protein results in levels of mice hematocrit as increased as if the same viral dose is injected intramuscularly or intradermally. Therefore, it is possible to conclude that intranasal administration is an efficient way to obtain therapeutic protein secretion in the bloodstream.

A. Vector Construction and Adeno-Associated Virus (AAV) 2/5 Purification

The hybrid 2/5 packaging construct was prepared as described above, by ligation of the fragment p600 ΔCAP and fragment AAV5-CAP. Plasmid pAd-ΔF6 was prepared as described above.

PAAV2.1-CMV-mEpo was constructed as follows: the mEpo coding sequence was cut NotI-HindIII from PCR2.1mEpo and cloned into pAAV2. 1 .CMV.LacZ cut in the same way.

Similarly, pAAV2.1CC10LacZ and pAAV2.2-CC10rhEpo were constructed by cutting pCF2.1 CC10 with Nhe-PstI and cloning the resulting 320 bp of the CC10 promoter into NheI-PstI-cut AAV2.1-CMVlacZ and pAV2.1CMV-rhEpo, respectively.

B. Virus Production

All recombinant virus stocks were produced helper-virus free in the following way. 293 cells were triple-transfected with the corresponding cis plasmid, packaging construct and Ad helper pF6. For production of AAV2/5 hybrid vectors, the corresponding AAV2 cis plasmid was used. Cells were harvested 72 hours after transfection, and recombinant virus was purified by three rounds of CsCl₂ banding. Titers were determined by real-time PCR.

C. Animal Studies

Five (5)—six week C57/BL6 mice were administered 1×10¹⁰ genomic copies of AAV2/5 either intranasally or via an alternative route for comparison as indicated. Sixty days after vector administration transgene expression was assessed either by β-galactosidase staining or by hemacrit measurements.

When lacZ expression was assessed in murine lungs after intranasal administration, as described, lacZ positive cells were evident in the upper airway epithelial cells and in the lower airway. Positive cells were present at the level of a bronchiole's wall as well as at the interalveolar septum level.

FIG. 1 provides the results observed from intranasal administration of 1×10¹¹ of either AAV2/5-CC10-rhEpo (rhEpo) levels in the bloodstream or AAV2/5-CC10-lacZ (β-galactosidase staining of lung) at 28, 60, and 90 days after vector administration. Analysis of hematocrit levels of the mice revealed that aerosolized AAV2/5 encoding mouse and human erythropoietin results in levels of hematocrit as increased as if the same viral dose is injected intramuscularly or intradermally (not shown).

All publications cited in this specification are incorporated herein by reference. While the invention has been described with reference to particularly preferred embodiments, it will be appreciated that modifications can be made without departing from the spirit of the invention. Such modifications are intended to fall within the scope of the claims.

What is claimed is:

1. A non-invasive method for obtaining pharmaceutically effective levels of a product in the bloodstream, said method comprising the steps of:
administering to a subject, by inhalation, a recombinant adeno-associated virus (AAV) comprising a transgene encoding a product under the control of regulatory sequences which direct expression of the product in lung cells transfected with the rAAV, whereby the expressed product is passed to the bloodstream from the lung cells.

2. The method according to claim 1, wherein the recombinant AAV is formulated in a liquid suspension for aerosol or spray delivery.

3. The method according to claim 1, wherein the recombinant AAV is administered at a dose of 1×10¹⁰ to 1×10¹⁵ genomic copies.

4. The method according to claim 1, wherein the recombinant AAV comprises AAV 5′ ITRs, a transgene and 3′ AAV ITRs in an AAV capsid protein.

5. The method according to claim 4, wherein the recombinant AAV comprises ITRs of an AAV serotype heterologous to the serotype of the AAV capsid protein.

6. The method according to claim 5, wherein the recombinant AAV comprises AAV2 5′ ITRs, a transgene and 3′ AAV2 ITRs in a capsid protein of AAV5.

7. The method according to claim 1, wherein the transgene encodes a secreted product selected from the group consisting of apolipoprotein E, erythropoietin, Factor IX, and Factor VIII.

8. The method according to claim 1, wherein the transgene encodes an antibody or a functional fragment thereof.

9. The method according to claim 1, wherein the transgene encodes a secreted protein having high affinity to presinillin.

10. A pharmaceutical kit for delivery of a secreted product, said kit comprising:
a suspension for aerosol or spray delivery of a predetermined dose by inhalation, said suspension comprising a recombinant AAV comprising a transgene encoding a secreted product and a physiologically compatible carrier.

11. The kit according to claim 10, further comprising a container for delivery of the predetermined dose.

12. The kit according to claim 11, wherein the container is designed for aerosol delivery of the dose.

13. The kit according to claim 11, wherein the container is designed for delivery by pump spray.

14. The kit according to claim 10, wherein the dose of recombinant AAV is 1×10¹⁰ to 1×10¹⁵ genomic copies.

15. The kit according to claim 10, wherein the recombinant AAV comprises AAV 5′ ITRs, a transgene and 3′ AAV ITRs in a capsid protein.

16. The method according to claim 15, wherein the recombinant AAV comprises ITRs of an AAV serotype heterologous to the serotype of the AAV capsid protein.

17. The method according to claim 16, wherein the recombinant AAV comprises AAV2 5′ ITRs, a transgene and 3′ AAV2 ITRs in a capsid protein of AAV5.

18. The pharmaceutical kit according to claim 10, wherein the transgene is apolipoprotein E.

19. The pharmaceutical kit according to claim 10, wherein the kit is used for treatment of hemophilia and the transgene is selected from the group consisting of Factor IX and erythropoietin.

20. The pharmaceutical kit according to claim 10, wherein the kit is used for treatment of diabetes and the transgene is an insulin protein.

21. The pharmaceutical kit according to claim 10, wherein the kit is used for the treatment and/or prevention of Alzheimer's disease and the transgene is selected from the group consisting of an anti-presinillin single chain antibody and a synthetic zinc finger transcription factor that dominantly represses the presinillin promoter.

22. The pharmaceutical kit according to claim 10, wherein the transgene encodes an antibody or functional fragment thereof.
